(19) United States
(12) Patent Application Publication
(10) Pub. No.: US 2016/0081846 A1
(43) Pub. Date: Mar. 24, 2016

(54) ARRANGEMENT FOR HEATING A PATIENT SUPPORT

(71) Applicant: MAQUET GMBH, Rastatt (DE)

(72) Inventor: Bernhard Katzenstein, Iffezheim (DE)

(21) Appl. No.: 14/927,029

(22) Filed: Oct. 29, 2015

Related U.S. Application Data
(63) Continuation-in-part of application No. PCT/EP2014/059620, filed on May 12, 2014.

(30) Foreign Application Priority Data
May 14, 2013 (DE) 10 2013 104 931.1

(51) Int. Cl. A61F 7/00; A61B 19/02

(52) U.S. Cl. CPC A61F 7/007; A61B 19/0248; A61B 2019/0255; A61B 2019/0298; A61F 2007/0094

(57) ABSTRACT

An apparatus is disclosed. The apparatus has a patient support cushion and a heating device that is integrated in the patient support cushion. The apparatus also has a temperature sensor that is integrated in the patient support cushion and a patient support cushion controller. The patient support cushion controller controls the heating device that is integrated in the patient support cushion based on an actual temperature value detected by the temperature sensor and a predetermined temperature value. The patient support cushion controller controls heating of the patient support cushion by the heating device based on a closed control loop that includes the heating device that is integrated in the patient support cushion and the temperature sensor that is integrated in the patient support cushion.

### CROSS REFERENCE TO RELATED APPLICATIONS

The present application is a continuation-in-part filed under 35 U.S.C. §111(a), and claims the benefit under 35 U.S.C. §§365(c) and 371 of PCT International Application No. PCT/EP2014/059620, filed May 12, 2014, and which designates the United States of America, and German Patent Application No. 10 2013 104 931.1, filed May 14, 2013. The disclosures of these applications are herein incorporated by reference in their entirety.

### TECHNICAL FIELD

The invention relates to an arrangement for heating a patient support, for example for heating cushion elements of a patient support of an operating table. The arrangement comprises a first cushion element and at least a second cushion element, each having an electric heating element integrated in the cushion element.

### BACKGROUND

During a medical treatment and during surgery, a patient's body temperature can decrease due to the ambient temperature which is lower than the body temperature during the treatment. Such a decrease of the body temperature, in particular of the core temperature, of the patient can lead to complications during or after the treatment or surgery. In order to avoid this, various heating devices for heating patients, or for keeping them warm, are known which keep the patient's loss of heat low during the treatment or surgery. Although the heating devices are typically not meant for increasing the patient's core temperature, heating systems can reduce undercooling of the body to a minimum.

Further, these heating systems are typically not operated permanently, in particular during preparation and after-treatment for surgery, for various reasons such that during this time the patient's core temperature can decrease. Common heating devices consist, for example, of heating pads which are positioned on the support surface pre-operatively. Adjustment of modular interchangeable components is more complicated with the additional heating pads than without such heating pads. Liquid circulating heating pads further have the disadvantage that they have a relatively heavy weight as they are at least partly filled with liquid. Further, irregular distribution of liquid in the heating pads has a negative influence on X-ray photographs and resting characteristics of the patient support.

Further, with heating pads placed as a supplement on the cushion elements, they are typically cleaned at least after an operation by disinfection, in addition to the subjacent cushion elements. This increases the time used for treatment of the patient support after an operation. Further, storage of the separate heating pads involves considerable storage room because heating pads and the supply pipes thereof usually are not folded or bent.

As an alternative to known heating pads, a further possibility involves placing the patient in a stream of warm air during the operation. Alternatively, radiation of the patient by heat radiators is possible. These solutions do, however, involve the persons carrying out the operation being subject to the heat radiation, too, which may not be desirable.

Heat output of known heating pads can, for example, be controlled at a separately arranged control unit fixed to an infusion stand.

The control unit can comprise an operating interface, a so-called user interface for manipulating the heating system comprised of a control unit and a heating pad.

From document WO 2008/110922 A2, a cushion is known with an integrated heating element as well as temperature sensors for detecting the temperature of the cushion. Further, a circuit integrated in the cushion is known, for transmitting the temperature values detected by the temperature sensors to an external control unit.

### SUMMARY OF THE DISCLOSURE

It is an object of the exemplary disclosed apparatus to provide an arrangement for heating for a patient support, for example for heating cushion elements for a patient support of an operating table, wherein simple and secure handling is possible.

As an alternative to one-piece heating pads or one-piece cushion elements with integrated heating elements, providing a plurality of cushion elements each of which has an electrical heating element integrated in the cushion element may allow for maintaining the adjustment function of components of a patient support composed of a plurality of components. Further, heat output can be adjusted in various regions of the patient support in a simple and variable manner, wherein the cushion-element control units control heat output of the heating element of the respective cushion element. Thereby a desired accuracy may be achieved regarding heat output of the desired amount of heat to the patient. Therein, the control loop for controlling heat output of the heating elements, or for controlling the temperature of the heating element/and or the cushion element may be completely integrated in the cushion element. Therein, the current flowing through the heating element may be set as the controlled variable, or pulse-width modulation of the supply voltage for supplying the heating element with electric energy may be effected. The actual temperature detected by the temperature sensor may serve as an actual value. This temperature sensor may be a passive temperature sensor such as, for example, a two-wire temperature sensor. PT100 sensor elements, PTC thermistors, PTC100 thermistors, and NTC thermistors may be, for example, temperature sensors. The central processing unit may have a user interface for operating the central processing unit, for example for setting the desired temperature values and/or heating output. Further, information such as error messages, status indications, actual and/or desired temperatures can be displayed via a display unit.

Based on the integration of the heating elements in the cushion elements, cleaning and disinfection efforts may be reduced as compared to the heating elements provided supplementary to the cushion elements. The efforts for disassembly and storage of heating pads and cushion elements may be reduced as compared to separate cushion elements and heating pads.

The heating output of the heating elements of the cushion elements may be dimensioned such that the heating output caused on the surface of the patient support has a maximum value in the range of 90 to 200 W/m², for example, a value smaller than 115 W/m² (e.g., between about 90 W/m² and about 115 W/m²). Thereby, a desired rapid heating of the cushion elements and heat supply to a patient contacting the cushion elements may be achieved.

The central processing unit may set the same desired temperature value for the two cushion-element control units, or may set an individual desired temperature value for each cushion-element control unit independently. This may allow for easy handling or individual heat output of the individual cushion elements such that the heat output can be systematically adapted to the patient and to the criteria of the treatment to be performed on the patient.

Further, each cushion-element control unit may transmit the actual temperature value detected by the temperature sensor to the central processing unit. For this purpose, a bidirectional data transfer between the central processing unit and the cushion-element control unit may be provided. Transfer of the detected actual temperature values to the central processing unit may allow for simple central temperature monitoring. Further, the transmitted actual temperature values can be displayed on a display unit of the central processing unit and/or error signals can be generated and output by the central processing unit. Further, the central processing unit may store or journalize at least a part of the actual temperature values transmitted by the cushion-element control units. The transmitted actual temperature values can be stored at a predetermined time interval, assigned to a respective current time. The predetermined time interval therein may be set to a value in the range of between 1 second and 5 minutes, for example to an interval of 10 seconds to 60 seconds. Thereby, the chronological sequence of the actual temperature values may be stored. This chronological sequence can be graphically displayed on a display unit of the central processing unit or on a further data processing unit connected thereto, such as a display unit of a treatment room or operating room central processing unit.

Each cushion-element control unit may include a protection circuit and/or a function to avoid overheating of the heating element and/or the cushion element.

Each cushion element may include at least one foam-material molded part. This may allow for comfortable and simple positioning of the patient. The shape of foam-material molded parts can be easily adapted. Further, the hardness of the foam-material molded parts may easily be adapted to the respective cushion element by suitably choosing the foam material applied or adopting suitable production methods.

At least the first one of the at least two cushion elements may have a first electric connection for supplying at least the electric energy used for the heating elements of the first cushion element and the second cushion element, and at least a second electric connection for providing the electric energy used for the heating element of the second cushion element. Thereby, the electric energy used for the second cushion element can be simply conducted through the first cushion element. Thereby the wiring effort for electric energy supply to the heating element and/or the cushion-element control units can be considerably reduced. Further, handling of the cushion elements can be considerably facilitated.

The first electric connection, in addition to serving for input of control signals and/or control data, may serve for transmitting the at least one desired temperature value to the cushion-element control unit integrated in the first cushion element. In this case, the second electric connection, in addition to serving for provision of control signals and/or control data, may serve for transmitting the at least one desired temperature value to the second cushion-element control unit integrated in the second cushion-element. Thus, the electric energy used for the electric heating elements as well as the control signals and/or control data to be transmitted from the central processing unit to the cushion-element control units can be transmitted via the same electric connections such that only one electric connector may be provided between the cushion elements for establishing the electric connections. Therein, transmission of the control signals and/or control data can also be performed via the same lines as the energy supply of the cushion elements. For example, the control data can be modulated onto the supply voltage, e.g. in terms of powerline data transmission.

The first electric connection and/or the second electric connection may be established by an electric connector with at least two electric contacts for electric energy supply for providing the heat conduction of the electric heating elements and two electric contacts for supply of the control signals or control data. Thus, the electric connections to the cushion elements may be established in a simplified way. As an alternative to the control signal transmission or control data transmission via the first and/or second electric connection, wireless transmission thereof, e.g. via infrared, wireless local area networks or Bluetooth data connections is possible.

The electric connection of the cushion elements may be serial and/or radial. This may allow for a simple connection of the cushion elements with the central processing unit as well as of the cushion elements with each other. The wiring effort for connecting the cushion elements with the central processing unit and the central energy supply can be considerably reduced thereby and handling of the cushion elements can be facilitated. Serial connection of at least two cushion elements for looping through supply lines for electrical energy supply and data lines for input of control data or for looping through control signals may be provided (e.g., a daisy chain). For example a number of hardware components which are connected in series may be provided, e.g. in bus systems (for example, for automation and automotive technology). Therein, the first component may be directly connected to the central processing unit. The other components each may be connected to their respective antecessor. A chain may be thus generated (e.g., a daisy chain). The components of the arrangement according to the invention may be the cushion-element control units and/or the heating elements of the cushion elements.

A bus system may be provided for transmission of data between the central processing unit and the cushion-element control units, with at least the desired temperature value or the desired temperature values being transmitted from the central processing unit to the cushion-element control units and/or the detected temperature values being transmitted from the cushion-elements control units to the central processing unit via said bus system. For example, a CAN bus system may be employed as a bus system. The control data and/or the control signals transmitted by the bus system may additionally or alternatively comprise error signals, program data, enable signals and/or further operating parameters of the central processing unit and/or the cushion-element control units. The control data and/or control signals can be transmitted by signals modulated onto the supply voltage. This may allow for a simple and cost-efficient connection of the cushion elements with the central processing unit for transmitting control data and control signals, respectively (e.g., as only the connection of the cushion elements with the supply voltage that is established). As an alternative, the control data and/or control signals may be transmitted via separate lines.

Another exemplary embodiment of the invention relates to an operating table having a patient support comprising at least a first adjustable component and a second adjustable component. The first component of the patient support may comprise the first cushion element, and the second component of the patient support may comprise the second cushion element. The cushion elements of the patient support can thereby be handled in a secure manner and can be easily installed by adopting the cushion elements according to the invention.

In the operating table, the first cushion element can be detachably connected to a supporting frame or a supporting panel of the first component, and the second component can be detachably connected to a supporting frame or a supporting panel of the second component. Thereby simple handling and connection of the cushion elements with the operating table is possible.

Further, in the operating table, the supporting frames and/or supporting panels may be electrically connected to each other for providing the electric energy used for the electric heating elements and for transmitting the at least one desired temperature value. The electric connection may be removable, for example through a suitable connector arrangement. For this purpose, the connector arrangement may be integrated in mechanical connectors for connecting the components such that the electric connection via the connector arrangement is automatically achieved when the mechanical connection is established.

The cushion elements may be connected with the supporting frame via at least a removable electric connection assembly or, alternatively, via an inductive connection for providing the electric energy used for the electric heating elements and for transmitting the at least one desired temperature value. This allows for simple installation and handling of the cushion elements according to the invention such that the cushion elements of the patient support can be easily heated. The operating table may have at least one storage battery at least for short-time energy supply of at least one of the electric heating elements and at least of the cushion-element control unit controlling the respective cushion element, which storage battery is integrated in the patient support, in the respective cushion element, in the central processing unit, in a transport carriage for transporting the patient support, and/or in an operating table column of the operating table. Thereby it is possible to enable heat supply to a patient resting on the patient support even if the operating table cannot be supplied with energy from a mains power supply (for example, when the patient support is transported separately from the operating table column).

As mentioned above, the cushion-element control units may control the heat output of the heating element of the cushion element in dependence on an actual temperature value detected by the temperature sensor and the predetermined desired temperature value. Control may include a process wherein the actual temperature value, which may be variable, is automatically kept constantly or approximately constantly at the desired temperature value. The actual temperature value may be determined by the temperature sensor as the measured value. In case of a discrepancy between the actual temperature value and the desired temperature value (e.g., control deviation), the heat energy output by the heating element of the respective cushion element may be adjusted in dependence on the determined control deviation such that actual temperature value approaches the desired temperature value. In general, control or controlling may involve a process wherein a variable quantity (actual temperature value) is continuously detected and compared with another variable quantity (desired temperature value) and is manipulated in the sense of approximation to the other variable quantity. The detected variable quantity may be a controlled quantity whereas the other variable quantity may be a command quantity. Control or controlling may include a feature of a closed loop, and the controlled quantity may continuously influence itself in the control loop.

The central processing unit may adjust the desired temperature value or the desired temperature values of the contact surface of the cushion element for contact with a patient depending on the patient's body temperature automatically in a preset temperature range of between about 0° C. and about 41° C. (for example, about 24° C.). In this respect, the temperature sensor may detect the temperature at the contact surface.

Further features and advantages of the invention are disclosed in the following description, which in connection with the enclosed Figures explains the invention in more detail with reference to embodiments.

### BRIEF DESCRIPTION OF THE DRAWINGS

FIG. 1 shows the upper portion of an operating table column with a patient support connected to the operating table column;

FIG. 2 shows a transport carriage with the patient support according to FIG. 1 separated from the operating table column;

FIG. 3 shows a center segment connected to the operating table column, and four further segments of the patient support to be connected to said center segment, wherein the cushion elements of the center segment and a further segment are shown separately;

FIG. 4 shows a schematic illustration of the structure of the cushion elements and the connection of the cushion elements with a central energy supply unit and with a central processing unit according to a first embodiment; and

FIG. 5 shows a schematic illustration of the structure of the cushion elements and the connection of the cushion elements with a central energy supply unit and with a central processing unit according to a second embodiment.

### DETAILED DESCRIPTION AND INDUSTRIAL APPLICABILITY

FIG. 1 shows the upper part of an operating table 10, which may include the upper portion of an operating table column 14 and a patient support 12 connected to the operating table column 14. In the present embodiment, the patient support 12 may comprise six components, namely two leg panels 16, 18, a central center panel 20, two torso panels 22, 24 and a head panel 26. In the present embodiment, the components 16 to 26 may be arranged so as to be pivotable to each other about rotational axes 28, 30, 32, 34 via electromotive drives. Further, the patient support 12 may be arranged so as to be pivotable about the longitudinal axis 38 and the transverse axis thereof via corresponding pivot drives of an operating table head 36 which forms the upper portion of the operating table column 14. The components 16 to 26 may each comprise a base panel and/or a base frame on which they have at least one cushion element with at least one heating element integrated in the respective cushion element (e.g., the at least one heating element may be formed integrally with the respective cushion element, so that the at least one heating element is for example an integral part of the respective cushion element). In the present embodiment, a heating pad integrated in each cushion element is shown as a dashed line. In the present embodiment, the head panel 26 may have a cushion without an integrated heating element. In the exemplary embodiment illustrated in the Figures, the cushion elements of the respective components are designated by the reference sign of the respective component 16 to 26 followed by a lowercase “a”, the heating pad integrated in the cushion element is designated by the reference sign of the respective component 16 to 26 followed by a lowercase “b”, and the supporting frame or supporting plate is designated by the reference sign of the respective component 16 to 26 followed by a lowercase “c”. The leg panel 18 may thus comprise the cushion element 18a, the heating pad 18b, and the supporting frame 18c.

The operating table 10 may comprise a central processing unit 42 which is connected to the cushion element 20a, for example via the supporting panel 20c or directly, through a connector arrangement. Further, the central processing unit 42 may be connected to the electric power system of an operating room via a connection. The cushion elements 16a to 24a may be electrically connected to each other by connectors (e.g., detachable cable bridges), such that both the supply voltage and control signals and/or control data is connected in a bidirectional manner between the central processing unit 42 and cushion-element control units integrated in each of the cushion elements 16a to 24a (e.g., the cushion-element control unit may be formed integrally with the respective cushion element, so that the cushion-element control unit is for example an integral part of the respective cushion element). Via a man-machine-interface, e.g. via a user interface 44 of the central processing unit 42, the heating function of the heating pads 16b to 24b integrated into the cushion elements 16a to 24a can be activated. At least one desired temperature value can be set, based on which the desired temperature value set at the central processing unit 42 (e.g., the actual cushion temperature detected by the temperature sensor integrated in each of the cushion elements 16 to 24a) is controlled. Interfering signals, error signals, and the actual temperature values detected by the respective temperature sensor may be transmitted to the central processing unit 42 by the bidirectional data connection, and may be recorded thereby together with the current time of detection.

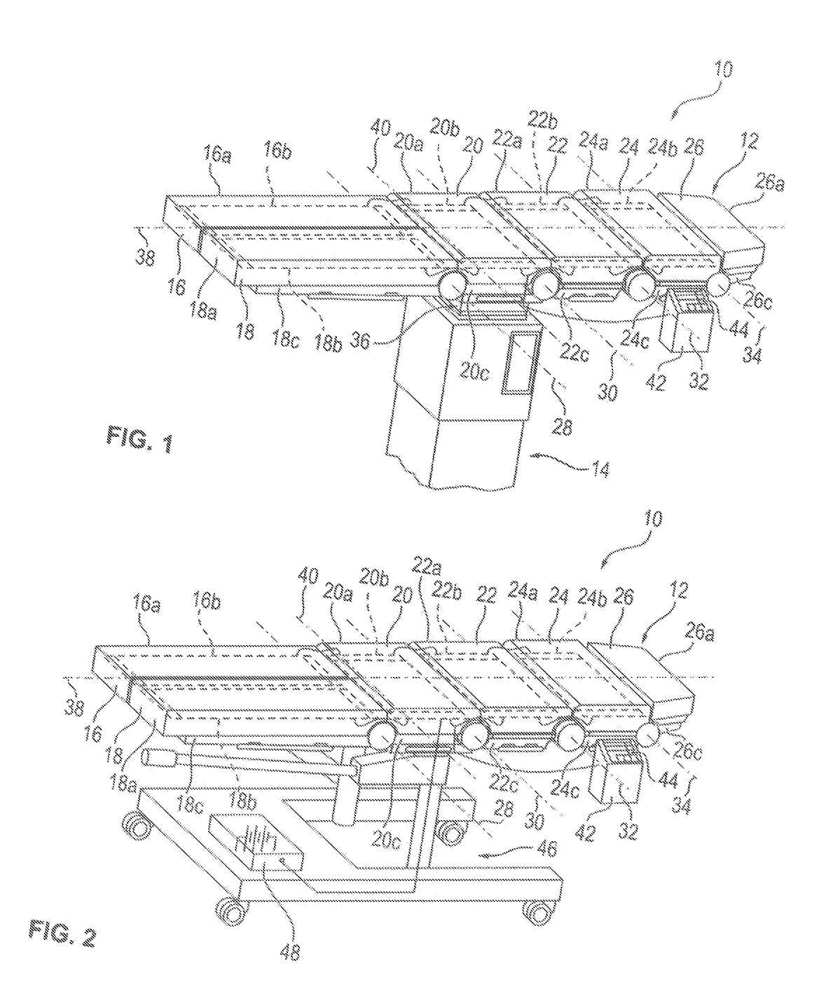

In FIG. 2, a transport carriage 46 is illustrated, together with the patient support 12 according to FIG. 1, separated from the operating table column 14. The energy supply of the heating pads 18b to 24b, of the cushion-element control units and the central processing unit 42 may not be connected through a mains power supply, for example as in case of the connection of patient support and operating table column 14, but through a storage battery 48 integrated in the transport carriage 46. Instead of or in addition to the storage battery 48, another battery can be provided. In another embodiment, an energy supply unit, like a battery or a storage battery may be provided in the central processing unit 42, wherein the battery or the storage battery may be replaceable.

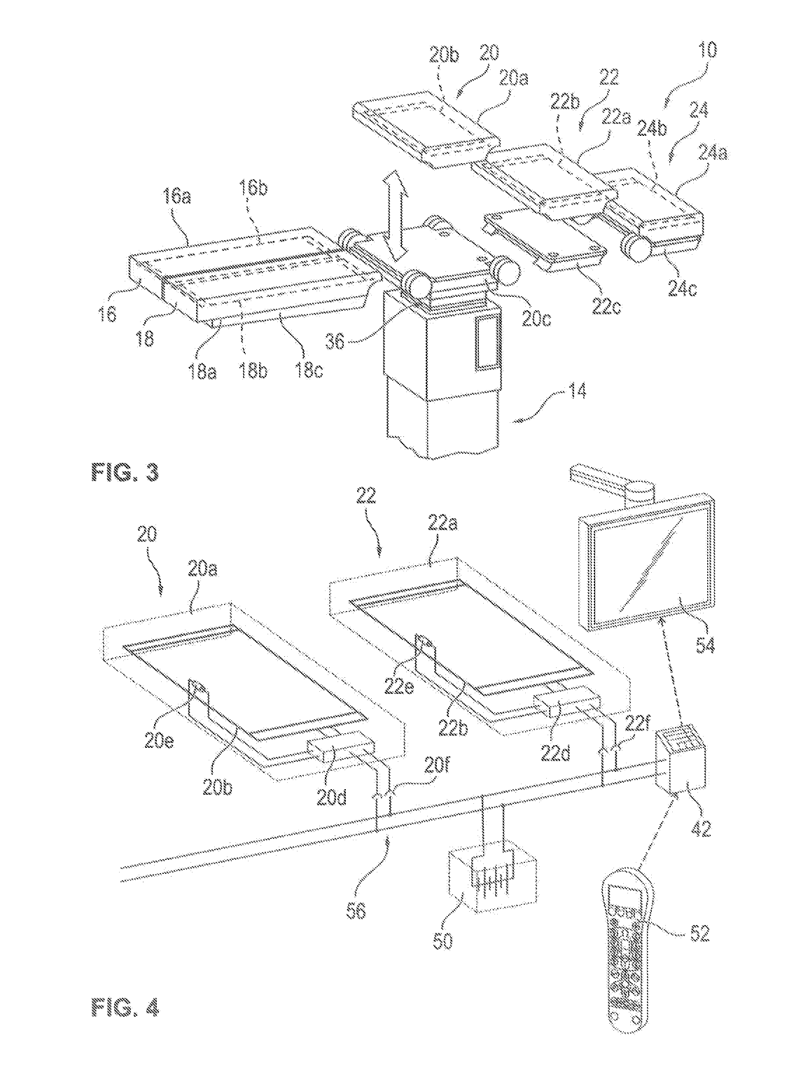

FIG. 3 shows the center panel 20 connected to the operating table column head 36 of the operating table column 14, wherein the cushion element 20a may be separated from the supporting panel 20c. The other components 16, 18, 22, 24, (e.g., the cushion elements 16a, 18a, 22a, 24a with the heating pads 16b, 18b, 22b, 24b) may be separated from each other and separated from the center panel 20. The cushion elements 20a, 22a of the components 20, 24 may be separated from the respective supporting panels 20c, 22c. The cushion elements 20a to 26a can be separated from the respective supporting panels 20c to 26c, or supporting frames, via detachable connections such that they can be easily cleaned and disinfected. Further, the cushion elements separated from the supporting panels 20c to 26c can be easily replaced by other cushion elements and can be easily transported and stored, for example in a stacked manner.

FIG. 4 shows a schematic illustration of the structure of the cushion elements 20a, 22a and of the connection of the cushion elements 20a, 22a with a central power supply unit 50 of the central processing unit 42 through a two-wire bus system. In all embodiments, as an alternative or in addition to the user interface 44 of the central processing unit 42, operator input is possible also via a wireless or a wired remote control 52, or via central control unit 54 which is connected to the central processing unit via either wireless or wired connection, wherein a plurality of control functions, such as light control, air-condition control, actuation of the operating table and further medical equipment in the operating room, can be activated and monitored by the central control unit.

Inside the cushion element 20a at least one temperature sensor 20e and one cushion-element control unit 20d may be arranged besides the heating pad 20c. Via a connector assembly 20f, the cushion-element control unit 20d may be connected to the two-wire bus system 56 through which control data are transmitted between the cushion-element control units 20d, 22d and the central processing unit and through which the supply voltage for the cushion-element control units 20d, 22d, the temperature sensors 20e, 22e, and the heating elements 20c, 22c is provided. The cushion elements 16a, 18a, 24a of the other components, like the cushions 20a, 22a, each comprise a cushion-element control unit 16d, 18d, 24d, at least one temperature sensor 16a, 18e, 24e, and at least one connector assembly 16f, 18f, 24f.

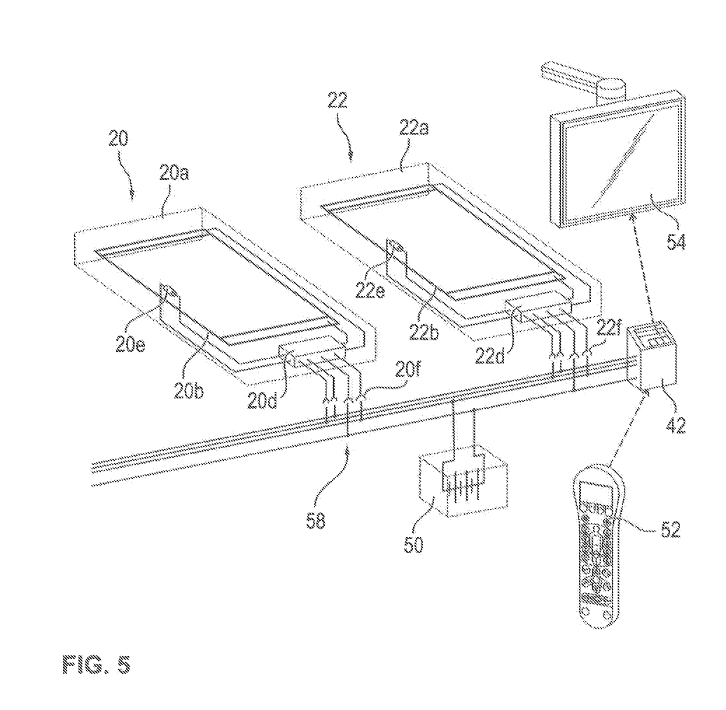

FIG. 5 shows a schematic illustration of an alternative structure of the cushion elements 20a, 22a, and of a connection of the cushion elements 20a, 22a with the central power supply unit 50 and the central processing unit 42 according to a second embodiment, as another exemplary embodiment in addition to the first embodiment according to FIG. 4. In this second embodiment, a four-wire bus system may be used instead of a two-wire bus system, wherein the control data or the control signals may be transmitted between the central processing unit 42 and the cushion-element control units 20d, 22d via separate lines such that these signal lines are decoupled from the power supply lines. This may allow for a higher transmission rate and less interference of the control signals and/or control data transmitted. For the sake of simplification, the cushion elements, temperature sensors, heating pads, cushion-element control units and the central processing unit are shown as designated by the same reference signs in the second exemplary embodiment as in the first exemplary embodiment.

In both the first exemplary embodiment according to FIG. 4 and the second exemplary embodiment according to FIG. 5, the other cushion elements 16, 18, 24 provided with heating pads may have the same structure as shown for the cushion elements 20a, 22a in FIGS. 4 and 5. Further, the cushion-element control units 16d, 18d, 24d of these cushion elements 16a, 18a, 24a may be connected with the respective bus systems 56, 58.

Further temperature sensors can be provided in order to detect the temperature at different positions of the respective cushion element 16a to 24a. For example, at least one sensor 16e to 24e may be located close to the surface, and a second temperature sensor may be located in the immediate vicinity of the heating pad, by which overheating of the respective heating pad can be detected.

Two-wire temperature sensors, such as, for example, a PT100 resistor element, PTC thermistors, NTC thermistors, PTC100 thermistors, or NTC100 thermistors may be used as temperature sensors. The heating power of the heating pads may be dimensioned such that the surface heating power of the cushion elements 16a to 24a is <=115 W/m².

The cushion-element control units 20d, 22d may control the actual temperature value to the pre-set desired temperature value in accordance with a closed loop control.

Depending on the embodiment, the central processing unit 42 can set the same desired temperature value for each of the cushion-element control units 16d to 24, or can set an individual desired temperature value for each cushion-element control unit 16d to 24d. Each cushion element 16a to 26a may comprise at least one foam-material molded part.

The temperature of the heating pad 16b to 24b may be detected by the temperature sensor positioned in the immediate vicinity of the heating pad 16b to 24b. The detected temperature value may be processed by a protection circuit and/or a protection function of the cushion-element control unit, wherein overheating of the heating pad 16b to 24b and/or the cushion element 16a to 24a may be detected and/or avoided by, for example, turning off the heating pads 16b to 24b.

The bus system employed may be a CAN bus system which provides for simple and secure data transmission between the central processing unit 42 and the cushion-element control units 16d to 24d. As an alternative to the above-mentioned wired solutions, data transmission may be performed via a wireless data transmission connection, like WiFi, Bluetooth or infrared. Further data transmission and/or power transmission can be made inductively between the respective cushion element 16a to 24a and the supporting panel 16c to 24c, or supporting frame, on which the cushion element 16a to 24a rests. For this purpose, the supporting frames and/or supporting panels 16c to 24c of the components 16 to 24 of the patient support 12 may be electrically connected to each other via corresponding connections.

The operating voltage supplied to the heating pads 16b to 24b can be lower than the mains voltage, for example an extra-low voltage. The central processing unit 42 and/ or the respective cushion-element control unit 16d to 24d can additionally comprise a storage battery which takes over power supply of the heating pad 16b to 24b in case the central processing unit 42 or the cushion-element control units 16d to 24d are separated from a mains power supply. The storage battery may be integrated in the respective control unit 42 (e.g., also cushion-element control unit 16d to 24d) and is charged via a corresponding charging circuit as long as the control unit 42 (e.g., also cushion-element control unit 16d to 24d) is connected to a mains power supply.

Instead of the heating pads 16b to 24b mentioned in the embodiments, other heating elements can be used, such as resistance-heating devices, preferably resistance-heating devices integrated in blankets.

The central processing unit 42 may automatically set the desired temperature value or the desired temperature values depending on the patient's body temperature within a predetermined temperature range, for example of 24° C. to 37° C.

What is claimed is:

1. An apparatus, comprising:
a patient support cushion;
a heating device that is integrated in the patient support cushion;
a temperature sensor that is integrated in the patient support cushion; and
a patient support cushion controller;
wherein the patient support cushion controller controls the heating device that is integrated in the patient support cushion based on an actual temperature value detected by the temperature sensor and a predetermined temperature value; and
wherein the patient support cushion controller controls heating of the patient support cushion by the heating device based on a closed control loop that includes the heating device that is integrated in the patient support cushion and the temperature sensor that is integrated in the patient support cushion.

2. The apparatus of claim 1, wherein the temperature sensor is selected from the group consisting of a PT100 sensor element, a PTC thermistor, a PTC100 thermistor, and an NTC thermistor.

3. The apparatus of claim 1, wherein the patient support cushion controller controls the heating device to produce a heating output at a surface of the patient support cushion of between about 90 W/m² and about 200 W/m².

4. The apparatus of claim 1, wherein the patient support cushion controller controls the heating device to substantially prevent an overheating of at least one of the patient support cushion and the heating device.

5. The apparatus of claim 1, wherein the patient support cushion includes a foam-material molded portion.

6. The apparatus of claim 1, wherein the patient support cushion is detachably connectable to a supporting frame of an operating table or a supporting panel of an operating table.

7. An apparatus, comprising:
a first patient support cushion;
a first heating device that is integrated in the first patient support cushion;
a first temperature sensor that is integrated in the first patient support cushion;
a first patient support cushion controller;
a second patient support cushion;
a second heating device that is integrated in the second patient support cushion;
a second temperature sensor that is integrated in the second patient support cushion;
a second patient support cushion controller; and
a main controller;
wherein the first patient support cushion controller controls the first heating device that is integrated in the first patient support cushion based on a first actual temperature value detected by the first temperature sensor and a first predetermined temperature value;
wherein the second patient support cushion controller controls the second heating device that is integrated in the second patient support cushion based on a second actual temperature value detected by the second temperature sensor and a second predetermined temperature value; and
wherein the main controller sets the first predetermined temperature value independently of the second predetermined temperature value.

8. The apparatus of claim 7, further comprising:
a power source that supplies power to the first heating device and the second heating device, wherein the main controller controls the power source; and
a storage battery that supplies power for short-time use to the first heating device and the second heating device, the storage battery being disposed at an operating table transport carriage or an operating table column.

9. The apparatus of claim 7, wherein the first predetermined temperature value and the second predetermined temperature value are substantially equal.

10. The apparatus of claim 7, wherein the first predetermined temperature value and the second predetermined temperature value are different.

11. The apparatus of claim 7, wherein the first and second predetermined temperature values are between about 24° C. and about 37° C.

12. The apparatus of claim 7, wherein:
the first patient support cushion controller transmits the first actual temperature value to the main controller;
the second patient support cushion controller transmits the second actual temperature value to the main controller; and
the main controller stores some or all of a plurality of first actual temperature values and second actual temperature values transmitted to the main controller from the first patient support cushion controller and the second patient support cushion controller.

13. The apparatus of claim 7, wherein:
the first patient support cushion includes a first connection that supplies the energy used by both the first heating device of the first patient support cushion and the second heating device of the second patient support cushion; and
the first patient support cushion also includes a second connection that supplies the energy used by the second heating device of the second patient support cushion.

14. The apparatus of claim 13, wherein:
the first connection transmits at least one of control signals, control data, and the first predetermined temperature value to the first patient support cushion controller integrated in the first patient support cushion; and
the second connection transmits at least one of control signals, control data, and the second predetermined temperature value to the second patient support cushion controller integrated in the second patient support cushion.

15. The apparatus of claim 13, wherein:
the first connector and the second connector each include at least one contact that supplies power for heating and at least one contact that transmits control signals or control data; and
the first patient support cushion is electrically connected to the second patient support cushion through one of a serial connection and a radial connection.

16. The apparatus of claim 7, further comprising a bus system that transmits data between the main controller and the first patient support cushion controller and the second patient support cushion controller;
wherein a plurality of first predetermined temperature values and a plurality of second predetermined temperature values are transmitted from the main controller to the first patient support cushion controller and the second patient support cushion controller; and
wherein a plurality of first actual temperature values and a plurality of second actual temperature values are transmitted from the first patient support cushion controller and the second patient support cushion controller to the main controller.

17. The apparatus of claim 7, wherein:
the first patient support cushion is part of a first adjustable component of an operating table;
the second patient support cushion is part of a second adjustable component of an operating table;
the first patient support cushion is detachably mechanically connectable to a support member of the first adjustable component of the operating table; and
the second patient support cushion is detachably mechanically connectable to a support member of the second adjustable component of the operating table.

18. The apparatus of claim 17, wherein:
the support member of the first adjustable component and the support member of the second adjustable component are electrically connected;
the first patient support cushion is connectable to the support member of the first adjustable component via one of a first detachable electric connector and a first inductive connection, the one of the first detachable electric connector and the first inductive connection providing the energy used for the first heating device and transmitting the first predetermined temperature value; and
the second patient support cushion is connectable to the support member of the second adjustable component via one of a second detachable electric connector and a second inductive connection, the one of the second detachable electric connector and the second inductive connection providing the energy used for the second heating device and transmitting the second predetermined temperature value.

19. An apparatus, comprising:
a plurality of patient support cushions;
a plurality of heating devices, wherein each of the plurality of heating devices is integrated in a respective patient support cushion of the plurality of patient support cushions;
a plurality of temperature sensors, wherein each of the plurality of temperature sensors is integrated in a respective patient support cushion of the plurality of patient support cushions;
a plurality of patient support cushion controllers, wherein each of the plurality of patient support cushion controllers controls a respective patient support cushion of the plurality of patient support cushions;
a main controller; and
an operator input device that provides operator input to the main controller;
wherein each patient support cushion controller controls each respective heating device that is integrated in each respective patient support cushion based on a respective actual temperature value detected by each respective temperature sensor and one of a plurality of predetermined temperature values;
wherein the main controller sets the plurality of predetermined temperature values, with each respective predetermined temperature value being set independently for each respective patient support cushion;
wherein each of the plurality of temperature sensors transmits respective actual temperature values to the main controller;
wherein the operator input device is selected from the group consisting of a wireless remote control, a wired remote control, and a central control unit that controls a plurality of medical devices of an operating room.

20. The apparatus of claim 19, wherein the main controller sets the plurality of predetermined temperature values based on a patient body temperature, with each of the plurality of predetermined temperature values being between about 24° C. and about 37° C.
